(19) United States
(12) Patent Application Publication
(10) Pub. No.: US 2023/0072155 A1
(43) Pub. Date: Mar. 9, 2023

(54) METHOD AND SYSTEM FOR INCORPORATING PATIENT INFORMATION

(71) Applicant: SimulConsult, Inc., Chestnut Hill, MA (US)

(72) Inventor: Michael M. SEGAL, Chestnut Hill, MA (US)

(21) Appl. No.: 17/797,559

(22) PCT Filed: Feb. 8, 2021
(86) PCT No.: PCT/US2021/017027

§ 371 Date: Aug. 4, 2022

Related U.S. Application Data
(60) Provisional application No. 62/970,847, filed on Feb. 6, 2020.

(51) Int. Cl. G16H 70/60; G16H 15/00

(52) U.S. Cl. CPC G16H 70/60; G16H 15/00

(57) ABSTRACT

The invention provides methods and related systems and computer readable media that may be used to aid a clinician in diagnosing a condition. The methods and systems identify and incorporate findings from patient information (such as the patient's electronic health record (EHR) or dictated text) and map them to findings in a list or database in a physical computing device, i.e., in a ODDS. The identification of findings may be carried out using various methods, such as natural language processing systems (NLP), either in real time or in advance.

### BACKGROUND

Rich information on patients may be available in charts of patients; however, detailed review of this information can be difficult, especially when the information is voluminous, or a rapid review is required. Thus, collecting such existing information may be time consuming and error prone when a clinician is seeking to make a diagnosis. There is a pressing need in the art to develop an accurate, efficient, and quantitative approach to retrieve the “findings” (e.g., signs, symptoms, family history, and test results). Such a machine-assisted chart review, using a clinician-in-the-loop process integrated with a computer-based diagnostic decision support tool that allows its use to compute the probability of various diseases, can thereby provide convenient and accurate diagnostic decision support to the clinician.

### SUMMARY OF THE INVENTION

Medical diagnosis would be improved if complete and exact data could be obtained in a structured form from patient information, e.g., the Electronic Health Record (EHR), if the process could be done in an automated way, e.g., using natural language processing (NLP), and then the data could be imported into a Diagnostic Decision Support System (DDSS). In this way the process of diagnosis would take less time without sacrificing quality, and often improving quality.

This goal has been difficult to achieve primarily for several reasons.

The main reason is that automated processes such as NLP produce too many false positives and false negatives to be used reliably in diagnosis without checking by humans, and much of the information produced is irrelevant.

Another reason is that such automated processes have difficulty interpreting factors important in making a diagnosis beyond mere presence of findings: information about absence of findings (“pertinent negatives”) and longitudinal data about time of presence (onset and disappearance of findings), and such judgments are often done better by humans.

Yet another reason is that decades of research in medical informatics have concluded that diagnosis is iterative, beginning with initial information and then adding further information based on the diagnostic possibilities. Humans are often better than computers at choosing the sequence of information leading to such iteration.

I have developed a system that allows a DDSS to process the results of NLP in a way that transcends the problems of false positives, false negatives and irrelevant information. Furthermore, it does so in a way that provides for the “clinician in the loop” to add information about presence, absence and onset, and exercise judgment about the iterative collection of information to arrive at a diagnosis. This results in a multi-step system that maximizes the signal-to-noise ratio, e.g., using the following steps:
1. Obtaining patient information, such as from the electronic health record (EHR). Using standards-based approaches (e.g., Fast Healthcare Interoperability Resources (FHIR) methods that meet the Health Level Seven International (HL7) standards), EHR patient information is retrieved and saved. This can be done as a regularly scheduled job, triggered by a clinic schedule, or on demand.
2. Locating mentions of findings using NLP, enrichment and special processing for numerical data and storing the context of these mentions. Standard NLP is used to associate patient information with the “concept codes” of medical terminology ontologies (e.g., the Unified Medical Language System (UMLS) and/or Human Phenotype Ontology (HPO)). This information then may be focused down to the small subset of findings of interest as defined by a curated set of concept codes paired with DDSS findings. The curation of these pairings is beneficial because findings used in a DDSS will map to many different UMLS concept codes, and the pairing results in collecting the full sense of a DDSS finding, while ignoring the large fraction of NLP-detected information not relevant to any DDSS finding. For example, the DDDS may ignore a substantial fraction of the concept codes that are not useful for diagnosis in a particular area of medicine, such as for genetic diagnosis ignoring all but 7,000 of the ˜1,000,000 UMLS concept codes, or for diagnosis more broadly ignoring all but 40,000 of the 1,000,000. Secondly, the system may enrich the results using direct searches for textual finding terms and synonyms in the DDSS database, augmenting situations where NLP has poor recognition such as for abbreviations (often deliberately ignored to reduce false positives in generic use). Thirdly, there can be separate methods of processing of numeric data to calculate the percentiles over time for key metrics used in diagnosis, e.g., height, weight, and head circumference. For each mention, the system collects the context, e.g., the text in which the finding was mentioned, date of the mention, patient age, and/or the identity and specialty of the author who recorded the observation. This context in which a concept code was identified constitutes an “object-oriented programming” software object, a “mention” object.
3. Formation of flagged finding software objects. The system collects mention objects associated with each finding in the DDSS, deduplicates the mentions, and forms a “flagged finding” object for each DDSS finding, containing all the unique mention objects associated with that finding. This answers the question “what findings were identified in the patient information?”
4. Favoring of relevant information by focusing on useful findings, done iteratively: useful findings may be computed by the DDSS based on the differential diagnosis (e.g., as described in U.S. Pat. No. 6,754,655, which is hereby incorporated by reference). This answers the question “what findings (whether mentioned in the patient information or not) are likely to be relevant for diagnosis?” The useful findings may be refreshed iteratively as more findings are added.
5. Favoring of reliable information through clinician assessment. The clinician reviews the flagged findings, and based on the contextual information in each mention in flagged findings, the clinician specifies which flagged findings to add to the patient's phenotype within the DDSS, whether these are pertinent positive or pertinent negative findings. This answers the question “what findings from the patient information are likely also to be reliable?”. The clinician supplements this information about the patient with information collected directly, such as on physical examination, using the same interface.
6. Ability to use the results of genomic analysis in combination with other findings. The system includes the ability to store, import, and process the annotated variants from genomic analysis in the DDSS together with other findings. Often this prompts an effort to collect more information about findings to confirm the clinical correlation, for which the flagged findings from the patient information can be very relevant.
7. “Finding list”. EHRs implement several lists, e.g., a “problem list”, “allergy list” and “medication list”. In this system, I add a human-readable output of a “finding list” that may include abnormal findings that were considered important for diagnosis, which included not only pertinent positive findings and their onsets, but also relevant “pertinent negative” findings asserted to be normal. The findings include standard ontology coding (e.g., HPO), regularly used by laboratories. Also supported are machine-readable standards-based outputs of findings (e.g., Phenopackets)
8. Return of Results report. Once a diagnosis has been made the clinician may use an automated, customizable Return of Results report to help the patient understand the diagnosis and care instructions. The system makes possible, in a standards-compliant way, to save a digitally signed version in the patient record for future reference by the patient and by the patient's other clinicians.

One embodiment of this invention is called the Genome-Phenome Archiving and Communication System with SimulConsult (GPACSS). The SimulConsult tool (SimulConsult, Chestnut Hill, Mass.) is a DDSS.

In general, the invention flags findings in the patient information and provides an opportunity for the user to specify which findings are convincingly present or absent, resulting in 4 types of findings.

| | |
|---|---|
| Flagged & specified | Flagged & not-specified |
| Not-flagged & specified | Net-flagged & not-specified |

The invention specifies what is flagged (top row) and allows the user to assess the information and specify the left column.

The invention can be used to improve the quality of diagnosis, cost of diagnosis, productivity of the clinician, and risk reduction. In particular, the invention can reduce the amount of time required for a clinician to review patient information and ultimately reach a diagnosis. It is designed in a way to give users complete control over private health information of their patients, within their data center.

In one aspect, the invention provides a method including the steps of providing a physical computing device having stored therein a plurality of candidate medical conditions and a list of findings, each of which is representative of clinical information about the medical conditions and wherein the findings in the list of findings are ranked as a function of the likelihood that the finding can disambiguate among the plurality of medical conditions; providing in the physical computing device one or more findings flagged as being identified from electronic patient information of a patient, wherein the physical computing device displays an indicator for any flagged finding in the list of findings; specifying in the physical computing device one or more flagged or not-flagged findings (e.g., at least one flagged finding) as being present or absent in the patient, wherein the physical computing device generates estimated probabilities of the medical conditions using the one or more findings specified as being present or absent; and outputting a candidate disease list of the medical conditions ranked by highest estimated probabilities.

In certain embodiments, the method further includes automatically reranking the findings in the list of findings as a function of the likelihood that the finding can disambiguate among the plurality of medical conditions changing as a result of changes in the list of findings specified by the user as being present or absent in the patient.

In certain embodiments, the method further includes identifying in the physical computing device one or more findings not identified from the electronic patient information as being relevant to diagnosis of the patient.

In certain embodiments, the method further includes displaying mentions of one of the flagged findings from the electronic patient information. The method may also aggregate multiple mentions of one of the flagged findings and/or eliminate duplicates of the same mention of one of the flagged findings prior to the displaying. In certain embodiments, the providing in the physical computing device of one or more flagged findings includes processing of numeric data to determine percentiles over time and/or processing clinical notes to identify contextual information for flagged findings. In certain embodiments, the method includes displaying the flagged findings in an integrated list with other findings not flagged as being in the electronic patient information; and/or displaying only the flagged findings in a standalone list. In certain embodiments, the flagged findings are identified using natural language processing (NLP) of the electronic patient information, either in real time or in advance, and/or using keyword searching, e.g., use of synonyms and/or abbreviations, of the electronic patient information, e.g., both are employed. In certain embodiments, ontology codes identified as being present in the electronic patient information are matched to one or more findings in the list of findings, optionally wherein one or more ontology codes identified as being present in the electronic patient information are not matched to any findings in the list of findings. In certain embodiments, at least one ontology code is matched to more than one finding; an ontology code from a parent, sibling, and/or child concept is matched to the one or more findings; and/or ontology codes from more than one ontology are matched to the one or more findings.

In certain embodiments, the method includes inputting in the physical computing device the onset timing and/or the timing of disappearance, of the finding.

In certain embodiments, the method further includes displaying contextual information (e.g., text from the electronic patient information) from the electronic patient information about each flagged finding, e.g., to aid the user in determining the reliability of the finding. The contextual information may allow for the determination of presence, absence, onset timing and/or the timing of disappearance of the flagged finding. In certain embodiments, the method further includes outputting a findings list useful in diagnosis (e.g., the findings list includes ontology codes in human readable and/or machine-readable formats; outputting a report, e.g., the Return of Results report; and/or saving a report in the electronic patient information. In certain embodiments, the electronic patient information includes dictation (including real time dictation and dictated notes) or an electronic health record.

In certain embodiments, the method further includes generating the pertinence of the findings in the list of findings and displaying the findings with an indicator of pertinence or outputting the list of findings ranked by pertinence. In certain embodiments, ranking the not-specified findings includes weighting the likelihood that a finding can disambiguate between a plurality of medical conditions, in some embodiments by a factor representative of a possibility that a disease can be treated effectively. In certain embodiments, the findings include genetic sequencing information associated with the patient including identification of one or more genetic variants, and for each of the one or more genetic variants, a measure of zygosity for the patient, wherein for each of said one or more genetic variants, a severity score is provided in the plurality of genetic findings or the computing device generates said severity score, and wherein estimated probabilities of the candidate diseases are generated using the severity scores for each of the one or more genetic variants.

In certain embodiments, the method includes importing notes, chart values, lab results, and/or metadata about the context, date, and clinicians making the observation.

In certain embodiments, the method further includes testing for a finding not identified in the electronic patient information and/or treating the patient based on the estimated probabilities of the medical conditions.

In another aspect, the invention provides a non-transitory computer readable medium having stored therein a plurality of candidate medical conditions; a list of findings, each of which is representative of clinical information about the medical conditions; and instructions for causing one or more processors to execute steps. The steps include:
(i) ranking findings in the list of findings as a function of the likelihood that the finding can disambiguate among the plurality of medical conditions;
(ii) identifying one or more findings from an output of a search of electronic patient information of a patient and flagging those findings in the list of findings;
(iii) displaying an indicator to a user for any flagged finding in the list of findings;
(iv) providing an interface for the user to specify one or more flagged or not-flagged findings as being present or absent in the patient;
(v) generating estimated probabilities of the medical conditions using the one or more findings specified as being present or absent; and
(vi) outputting a candidate disease list of the medical conditions ranked by highest estimated probabilities.

In certain embodiments, the instructions further include automatically reranking the findings in the list of findings as a function of the likelihood that the finding can disambiguate among the plurality of medical conditions changing as a result of changes in the list of findings specified by the user as being present or absent in the patient. In certain embodiments, the instructions further include displaying to the user mentions of one of the flagged findings from the electronic patient information, e.g., where the instructions further include aggregating multiple mentions of one of the flagged findings and/or eliminating duplicates of the same mention of one of the flagged findings prior to the displaying.

In certain embodiments, the search of the electronic patient information includes processing of numeric data to determine percentiles over time or processing clinical notes to identify contextual information for findings. In certain embodiments, step (iii) includes displaying the flagged findings in an integrated list with other findings not flagged as being in the electronic patient information; and/or displaying only the flagged findings in a standalone list. In certain embodiments, the search uses natural language processing (NLP) of the electronic patient information, either in real time or in advance and/or using keyword searching of the electronic patient information.

In certain embodiments, the medium further has stored therein a set of curated ontology codes from the search of the electronic patient information that match with one or more findings in the list of findings, e.g., wherein the set of curated ontology codes includes codes from more than one ontology.

In certain embodiments, the instructions further include searching the electronic patient information for keywords and/or abbreviations to identify findings. In certain embodiments, the instructions further include displaying contextual information from the electronic patient information about each flagged finding. In certain embodiments, the instructions further include outputting a findings list useful in diagnosis; outputting a Return of Results report; and/or saving a report in the electronic patient information. In certain embodiments, the instructions further include generating the pertinence of the findings in the list of findings and displaying the findings with an indicator of pertinence or outputting the list of findings ranked by pertinence.

In certain embodiments, ranking the not-specified findings includes weighting the likelihood that a finding can disambiguate between a plurality of medical conditions by a factor representative of a possibility that a disease can be treated effectively. In certain embodiments, the findings include genetic sequencing information associated with the patient including identification of one or more genetic variants, and for each of the one or more genetic variants, a measure of zygosity for the patient, wherein for each of said one or more genetic variants, a severity score is provided in the plurality of genetic findings or the instructions further include generating the severity score, and wherein estimated probabilities of the candidate diseases are generated using the severity scores for each of the one or more genetic variants.

In another aspect, the invention provides a system including a physical computing device including one or more processors, a network communication interface, and one or more computer readable memories having stored therein a plurality of candidate medical conditions; a list of findings, each of which is representative of clinical information about the medical conditions; and instructions. The instructions, when executed by the one or more processors, cause the system to
(i) rank findings in the list of findings as a function of the likelihood that the finding can disambiguate among the plurality of medical conditions;
(ii) identify findings in an output of a search of electronic patient information of a patient and flag those findings in the list of findings;
(iii) display an indicator for any flagged finding in the list of findings;
(iv) provide an interface for a user to specify one or more flagged or not-flagged findings as being present or absent in the patient;
(v) generate estimated probabilities of the medical conditions using the one or more findings specified as being present or absent; and
(vi) output a candidate disease list of the medical conditions ranked by highest estimated probabilities.

In certain embodiments, the instructions further cause the system to automatically rerank the findings in the list of findings as a function of the likelihood that the finding can disambiguate among the plurality of medical conditions changing as a result of changes in the list of findings specified by the user as being present or absent in the patient.

In certain embodiments, the instructions further cause the system to display to the user mentions of one of the flagged findings from the electronic patient information, e.g., wherein the instructions further cause the system to aggregate multiple mentions of one of the flagged findings and/or to eliminate duplicates of the same mention of one of the flagged findings prior to the displaying.

In certain embodiments, the search of the electronic patient information includes processing of numeric data to determine percentiles over time or processing clinical notes to identify contextual information for findings. In certain embodiments, (iii) includes displaying the flagged findings in an integrated list with other findings not flagged as being in the electronic patient information; and/or displaying only the flagged findings in a standalone list.

In certain embodiments, the search uses natural language processing (NLP) of the electronic patient information, either in real time or in advance and/or using keyword searching of the electronic patient information.

In certain embodiments, the one or more computer readable memories has further stored therein a set of curated ontology codes from the search of the electronic patient information that match with one or more findings in the list of findings, e.g., wherein the set of curated ontology codes include codes from more than one ontology.

In certain embodiments, the instructions further cause the system to search the electronic patient information for keywords and/or abbreviations to identify findings. In certain embodiments, the instructions further cause the system to display contextual information from the electronic patient information about each flagged finding. In certain embodiments, the instructions further cause the system to output a findings list useful in diagnosis; output a report, e.g., Return of Results report; and/or save a report in the electronic patient information. In certain embodiments, the instructions further cause the system to generate the pertinence of the findings in the list of findings and display the findings with an indicator of pertinence or output the list of findings ranked by pertinence.

In certain embodiments, ranking the not-specified findings includes weighting the likelihood that a finding can disambiguate between a plurality of medical conditions by a factor representative of a possibility that a disease can be treated effectively.

In certain embodiments, the findings include genetic sequencing information associated with the patient including identification of one or more genetic variants, and for each of the one or more genetic variants, a measure of zygosity for the patient, wherein for each of said one or more genetic variants, a severity score is provided in the plurality of genetic findings or the instructions further cause the system to generate the severity score, and wherein estimated probabilities of the candidate diseases are generated using the severity scores for each of the one or more genetic variants.

In certain embodiments, the electronic patient information includes dictation (including real time dictation and dictated notes) or an electronic health record.

### BRIEF DESCRIPTION OF THE DRAWINGS

FIG. 1: Flowchart of the methods and systems of the invention.

FIG. 2: The finding of myopia is listed as #1 in the useful findings for a patient with Marfan syndrome and is shown in the DDSS with a flag denoting that myopia is mentioned in the EHR on 2 different dates. The flag button was clicked, and information about the 2 mentions of myopia is displayed.

### DETAILED DESCRIPTION

The invention provides methods and related systems and computer readable media that may be used to aid a clinician in diagnosing a condition. The methods and systems identify and incorporate findings from patient information (such as the patient's electronic health record (EHR) or dictated text) and map them to findings in a list or database in a physical computing device, i.e., in a DDDS. The identification of findings may be carried out using various methods, such as natural language processing systems (NLP), either in real time or in advance, by:
(i.) mapping the meaning attached to the findings in the physical computing device's database to a plurality of standard ontology concept codes in a way that minimizes false negatives in recognizing findings present in patient information by encoding in the physical computing device a particular, rich array of synonyms and parent, sibling, and child ontological concept codes for each finding in the physical computing device's database, specifically optimized for the purpose of diagnosis, and drawn from standard ontologies, such as UMLS and HPO;
(ii.) enriching the ability to identify findings in the patient information by directly searching for key words, synonyms, or acronyms for each finding of the physical computing device's database.

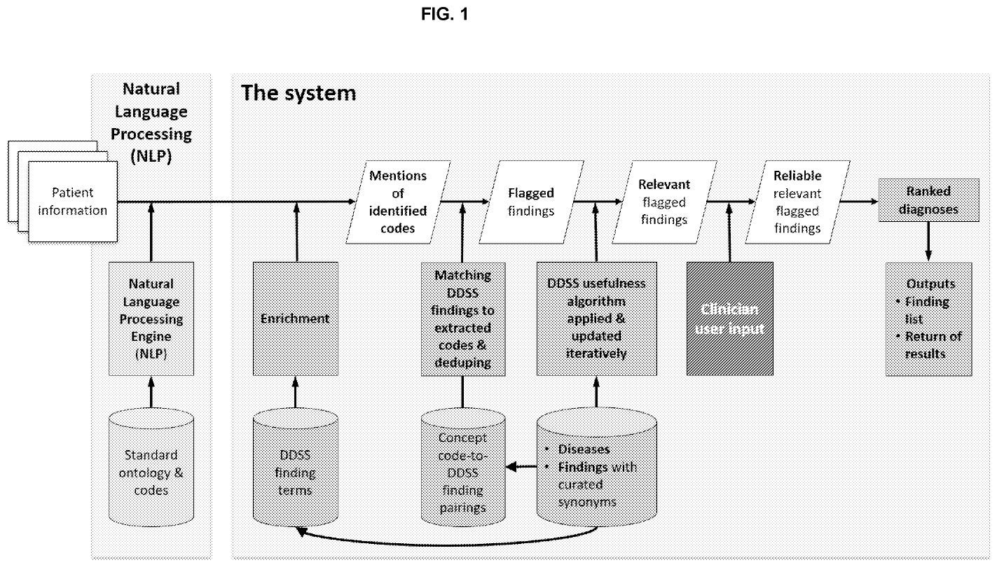

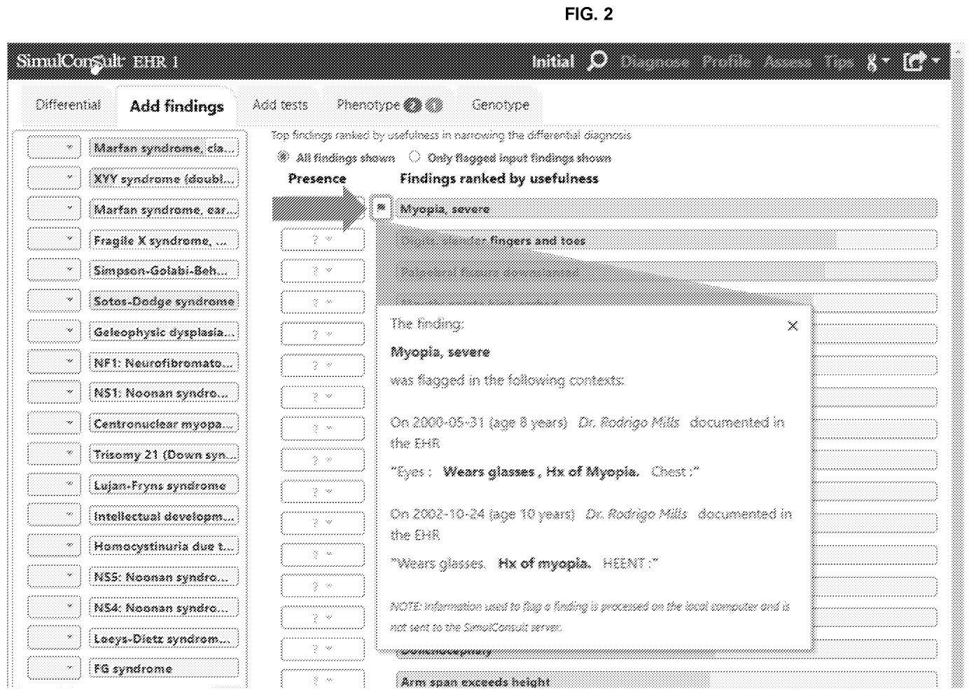

Based on the mapping, the methods and systems determine for each finding in the database, whether the finding is mentioned (as either present or absent) in the patient information. The methods and systems may also display an indicator, e.g., a flag, that a particular finding was mentioned in the patient information and/or a list of all such findings mentioned in the patient information. For each flagged finding, contextual information about each mention of a finding in the patient information may also be gathered and displayed, such as the date of the note where the finding was mentioned, the clinician who signed the note, and sufficient surrounding text, so that the user can assess whether the information is reliable, and links back to the original source information to make reading in more depth simpler and faster. A flowchart of the methods and systems is shown in FIG. 1, an example of a flagged finding and the excerpted patient information in its various mentions and the flag button used to display it is shown in FIG. 2.

In aiding diagnosis, the DDDS provides a list of a plurality of medical conditions, e.g., ranked by probability based on findings entered into the DDDS. Findings (whether or not mentioned in the patient information) can be ranked as a function of the likelihood that a finding can disambiguate among the plurality of medical conditions (i.e., “usefulness” as described in U.S. Pat. No. 6,754,655), whereby a clinician can employ the ranked findings to identify a finding, where commenting on the absence, presence, and/or timing of onset of that finding is likely to be relevant and lead to a correct diagnosis of the patient's medical condition. The various displays that include flagged findings may include:
(i.) avoiding the clutter and distraction of non-relevant positive findings from the patient information (e.g., a fever known to be related to the flu the patient had 3 months ago versus a fever that might be relevant for some of the potential diagnoses under consideration), by using the usefulness ranking shown in FIG. 2 to demote less useful findings to lower in the list (and sometimes later screens);
(ii.) displaying only the flagged findings in a standalone list, e.g., ranked by usefulness; and/or
(iii.) displaying the flagged findings in other integrated lists with those not found in the patient information, e.g., in a profile of a disease.

The DDSS uses findings specified by clinicians as being present or absent to offer a differential diagnosis and suggest other findings useful in making a diagnosis, thus providing guidance for clinicians in prioritizing further evaluation of the patient in an iterative manner. The DDSS uses not only findings that are present, and their time course, but also pertinent negatives. It can also import annotated genomic variant tables and interpret the results in the clinical context (Segal M M, et al. J Child Neurol. 2015 30:881-8; Segal M M, et al. Orphanet J Rare Dis. 2020 Jul. 22; 15(1):191). Once one or more findings from the patient information have been included, the DDDS may iteratively update the probabilities of the list of medical conditions. Such updating of the list of probable medical conditions, in turn, causes a re-ranking of the usefulness of findings. The clinician may also order additional tests, e.g., for useful findings not present in the patient information.

As shown in FIG. 1, findings in the DDSS are flagged as to whether they occurred in patient charts using NLP to identify concept codes and record surrounding text, as illustrated in FIG. 2. The NLP may be based on the open-source Apache cTAKES 4.0 clinical NLP platform (Savova et al. Journal of the American Medical Informatics Association 17.5 (2010): 507-513). The identified codes may then be used to flag DDSS findings based on a curated list of paired DDSS findings and standard ontology, e.g., UMLS or HPO, concept codes. The MedGen interface (https[://]www.ncbi.nlm.nih.gov/medgen) may be used for UMLS codes, and the interface at https[://]hpo.jax.org/app/may be used for HPO codes.

A key architectural component of the system is bridging the gap between the meaning of findings in the DDSS and the standard ontology concepts identified in the EHR.

Granularity: In a DDSS there is value to terminology that is “Mutually Exclusive and Collectively Exhaustive” (MECE), in contrast to the usual approach to building ontologies in which there is value in representing concepts at many levels of granularity. A MECE set of findings is important in a DDSS both for consistent curation of information at the same level of granularity and for probability calculations that are consistent for all diseases, allowing ranking of disease probabilities. As a result of this granularity issue, there is not a “one-to-one” mapping between MECE findings and the concepts in ontologies such as UMLS and HPO. Since different applications would make different granularity choices based on their own requirements, these specifications of meaning are best specified in the DDSS.

Effect of false negatives and false positives: When a clinician is using a DDSS, minimizing false positives of findings from the patient information is not of paramount importance if the clinician can reject such errors when reviewing mentions of a finding. This is especially true if such findings are already ranked by usefulness, thus filtered by demoting non-relevant findings to be lower in the list. In contrast, minimizing false negatives is very important since such errors could result in overlooking important information in the EHR.

For these reasons the DDSS preferably specifies the ontology terms that should be used for flagging each of its findings. The simplest way of specifying such granularity in a pure branching tree ontology would be specifying a node in the tree and automatically incorporating everything underneath or above, depending on the application. But medical terminology is far more complex, with a term often having more than one parent. As an example, in HPO, the concept “facial palsy” (HP:0010628) has 4 different parent concepts:
Abnormality of the seventh cranial nerve (HP:0010827)
Cranial nerve paralysis (HP:0006824)
Muscle weakness (HP:0001324)
Weakness of facial musculature (HP:0030319)

The difficulty with one-to-one pairing becomes even more difficult for “bundled terms” that include many other concepts, e.g. abnormal gait, which can result from many different causes.

Accordingly, each DDSS finding may list multiple concept codes, and a list of all such pairings is provided to a software module interacting directly with the patient information. This module analyzes patient information text and sends to the DDSS a list of flagged findings, with context information as that allows the clinician to assess each mention of the finding.

A standard ontology, such as UMLS, is like the English language in having many sources that are combined into one framework. This offers huge richness and interoperability, but often includes multiple classification systems and duplicate entries. As an example, pairing a DDSS finding of muscle cramps with UMLS concept codes requires locating a minimum of 2 “Sign or Symptom” codes in different branches of the UMLS ontology:
Muscle cramps (C0037763 with HP:0003394)
Cramp (C0026821 with no HPO code)

Both concepts are in the UMLS under “Abnormality of the musculature”. The first is on the branch “Abnormality of muscle physiology”. The second is on a different branch, “Abnormality of muscle morphology” in the sub-branch “Muscular Diseases” (diseases are a subject not typically covered in the HPO).

Many other difficulties in locating the totality of relevant concepts exist, including findings with two concepts with identical names in the same tree. One example from UMLS is alacrima 00344505 and alacrima 04012597, both as diseases under Decreased lacrimation 00235857. Another example from UMLS is the same idea represented as both a disease and a finding: vitiligo 00042900 as a disease with no HPO code and vitiligo 03277701 as a finding with HP:0001045, appropriate because in some situations one might want to consider vitiligo as a disease and in others as a finding occurring in another disease.

Process of assigning ontology codes: To overcome these issues, the DDSS may be configured to allow multiple standard ontology, e.g., UMLS and HPO, concepts for each of its findings and may use the following strategies to find the parent, child, sibling and synonymous concepts to attach to each DDSS finding. This may be performed with the following 2 goals: (1) representing the MECE nature of the DDSS findings and (2) minimizing false negatives in identifying the DDSS findings in the EHR.

To deal with the issue of granularity and the need to explore parent, sibling, child and synonym relationships, the following process of manual curation may be adopted for beginning with an HPO code and choosing all UMLS codes relevant to our MECE and false-negative goals.
Browsing to parents, siblings and children of the HPO code may be done first to look for concepts to attach to DDSS findings
   Parents: Parent concept codes may be included, for example the “Disease or Syndrome” “iron overload” 00282193 may be included for the many DDSS tissue iron overload findings such as “MRI: hepatic iron content increased”. The purpose of using such a parent concept is to reduce false negatives in NLP by signaling to the clinician that a mention of iron overload is likely a description of one or more specific iron overload findings. This results in some parent concepts being assigned to more than one DDSS finding. Because of the multiple parent capability of HPO, exploring for such relationships may be done first in HPO before looking for corresponding UMLS concepts.
   Siblings: Sibling concept codes may also be included. For example, widely spaced teeth (01844813) and oligodontia (04082304) are concepts appropriately considered as distinct in an ontology, but if either concept is found in an EHR, the clinician should consider whether this means the other. In this way, a DDSS can be MECE, yet also take the risk of a false positive in flagging concepts likely to coexist because the clinician can make the proper judgment when shown the mention of the flagged finding in the EHR.
   Children: Child concepts may be included when terminology ontologies were more granular than the MECE approach used for findings in the DDSS.
Using UMLS codes listed on HPO pages to locate corresponding UMLS pages, but these UMLS codes are sometimes missing.
Browsing to parents and children of the UMLS concept, limited by the lack of multiple parent support on the MedGen UMLS interface.
Text search in HPO using not only the finding name but the synonyms and explanatory terms collected by the DDSS curators and stored in the DDSS.
Using the MedGen interface to check for “round tripping”, i.e., checking whether the page for a UMLS concept linked back to the HPO page. A lack of round tripping typically means that another UMLS code exists that did link to the HPO code.
Text search in the MedGen UMLS interface to find UMLS codes not identified with the approaches above. In in many situations, searches using words in the sought UMLS concept may fail. However, in such situations searching for the HPO code may work, so such searches may be done when round-tripping failed.

The system may also supplement the NLP because of known gaps in NLP coverage. For example, NLP typically does not currently recognize two and three letter acronyms (e.g., “dtr” for deep tendon reflexes) regularly used in patient charts and certain other common concepts (e.g., tall is interpreted as “T-cell Acute Lymphoblastic Leukemia” rather than “tall stature”). So, the NLP concept code identification may be enriched by methods of direct search for text in the DDSS database of the terms and their synonyms for findings. As for search with ontology concept codes, the resulting mentions may be de-duplicated before sharing with the clinician.

A post-processing application may use the curated codes and the NLP pipeline output to flag DDSS findings according to the list of finding pairings provided by the DDSS. As discussed above, some concepts will flag more than one DDSS finding.

The methods and systems may be launched, e.g., from within an EHR or a software module that communicates with the EHR, in a way that automatically pulls in what has been read from the patient information and/or may save various reports, including reports that automatically share the relevant codes of the findings, e.g., codes from standard ontologies, where the findings are those the clinician has chosen to narrow down the differential diagnosis or reach a final diagnosis.

In certain embodiments, no confidential or identifying information needs to be transmitted to the system for analysis, e.g., no Patient Health Information (PHI), as defined under the US Health Insurance Portability and Accountability Act (HIPAA) and follow-on legislation, need be shared with the DDSS server, in order to accomplish its objectives.

The methods and systems may also include generating the pertinence of the findings specified by the user as being present or absent and outputting a list of such findings ranked by pertinence, a measure of the estimated contribution of that finding to driving the differential diagnosis (e.g., as described in U.S. Pat. No. 9,524,373, which is hereby incorporated by reference), making clear which findings are most strongly driving the most highly ranked diagnoses, and thus findings for which it is most important for the clinician user to be sure of their accuracy. The findings specified may also include genetic sequencing information associated with the patient, e.g., identification of one or more genetic variants, and for each of the one or more genetic variants, a measure of zygosity of such variants for the patient, wherein for each of said one or more genetic variants, a severity score is provided in the plurality of genetic findings or the computing device generates the severity score, and wherein estimated probabilities of the candidate diseases are generated using the severity scores for each of the one or more genetic variants (e.g., as described in U.S. Pat. No. 9,524,373).

Systems and Media

The invention also provides systems to carry out the methods of the invention and non-transitory computer readable media having stored therein instructions and data for carrying out the methods of the invention. Systems include a physical computing device with one or more processors, a network communication interface, and one or more computer readable memories to store data and instructions for carrying out the methods of the invention.

The physical computing device may be implemented in any suitable manner. For example, it may reside on a single server or computer or be distributed across multiple computers or servers, e.g., in a cloud architecture. The system may be accessed by a standard desktop or terminal by a dedicated program or webpage. The system may also be accessed via a mobile application. The system may be accessed within an EHR or another program or may be accessed by dedicated program that communicates with a source of patient information. The system may interact in a completely RESTful manner with a server that does not retain any information about the patient between client requests, which occur many times in a typical session.

The network communication interface may allow communication between the physical computing device and the user and/or a source of electronic patient information, e.g., an electronic health record. The interface may also allow communication between network components of the physical computing device.

The network communication interface may also be implemented as several different components, e.g., one for communication with the user and one for communication with sources of electronic patient information. Any standard network communication protocol may be employed, e.g., Transmission Control Protocol (TCP), Internet Protocol (IP), Global System for Mobile Communications (GSM) based cellular network, Wi-Fi, Bluetooth, and Near Field Communication (NFC). Connections may also be wired, wireless, or a combination thereof.

Any suitable computer readable memory or non-transitory computer readable medium may be employed in the physical computing device. Such memories and media include magnetic disks, optical disks, organic memory, and any other volatile (e.g., Random Access Memory (RAM), flash, and EEPROM) or non-volatile (e.g., Read-Only Memory (ROM)) mass storage system readable by the one or more processors. The memory or medium includes standalone or cooperating or interconnected memories or media, which may be distributed among multiple interconnected computers or servers that may be local or remote. In one embodiment, the data are stored with one or more encryption and/or security methods.

A system may also include any other components necessary for operation, e.g., displays, switches, and routers.

### Example

The following provides a non-limiting example of one implementation of the invention.

SimulConsult DDSS, a commercial product used by clinicians for assistance in making diagnoses, currently focused on complex diagnostic decision making in genetics, neurology, and rheumatology (Segal M M. Appl Transl Genom. 2015; 6: 26-27; Segal M M, et al. J Child Neurol. 2015 30:881-8; Segal M M, et al. J Child Neurol. 2014 29:487-492; Segal M M et al. Pediatric rheumatology online journal. 2016; 14:67) was used.

UMLS codes were used in order to utilize the cTAKES NLP system, which provided comprehensive high throughput phenotyping but did not have native support for HPO. The process began with 1,189 of the core clinical findings in the DDSS.

For these 1,189 DDSS findings, a total of 6,619 UMLS concept codes were assigned, an average of 5.6 per DDSS finding. High numbers of UMLS codes were required in situations of a concept being represented in many sources for the UMLS terminology, such as “developmental delay”. High numbers of UMLS concept codes were also assigned for DDSS findings with many UMLS child codes, such as for bones of the fingers and toes. Matching was particularly straightforward when a term from the MECE “Human Malformation Terminology” (Allanson et al. Am J Med Genet A. 2009; 149A: 2-5) already existed.

The result of this mapping and integration is shown in FIG. 1. The DDSS display (FIG. 2) shows a set of potential findings to enter for the patient, ranked in order of usefulness, defined as the ability to change the differential diagnosis in a way that prioritizes treatable diseases. The findings identified from the EHR and flagged by the system are denoted with flags, thereby providing an indication of which findings likely to be relevant are commented on in the EHR. Clicking such a finding displays the one or more mentions of the finding in the EHR. The display includes the sentence from which the finding was identified as well as the previous and subsequent sentence. Also shown is the date, patient age, and the clinician who entered the information. The display includes language to reassure the user that in flagging this finding, the SimulConsult server does not receive any of the information about this mention of the finding in the EHR; all it receives is a list of potential positive or negative findings, in the same anonymized manner in which it receives a list of the patient's actual findings. The clinician then assesses the finding and decides if it is reliable, and if so, enters into the DDSS the presence or absence of the finding, and onset information, specifying this using the components in FIG. 2 bearing the “?” symbol. By commenting on presence or absence for various flagged and non-flagged findings, the clinician provides a description of reliable and relevant findings (FIG. 1), both flagged as being in the EHR and those added by the clinician from other sources, such as physical information of the patient who is in the same room. This information is synthesized by the DDSS to offer advice on likely diagnoses (FIG. 2, diseases listed on left) and further testing.

The innovations described here provide rapid access to useful information in the EHR.

Other embodiments are in the claims.

What is claimed is:

1. A method comprising the steps of:
(a) providing a physical computing device having stored therein a plurality of candidate medical conditions and a list of findings, each of which is representative of clinical information about the medical conditions and wherein the findings in the list of findings are ranked as a function of the likelihood that the finding can disambiguate among the plurality of medical conditions;
(b) providing in the physical computing device one or more findings flagged as being identified from electronic patient information of a patient, wherein the physical computing device displays an indicator for any flagged finding in the list of findings;
(c) specifying in the physical computing device one or more flagged or not-flagged findings as being present or absent in the patient, wherein the physical computing device generates estimated probabilities of the medical conditions using the one or more findings specified as being present or absent; and
(d) outputting a candidate disease list of the medical conditions ranked by highest estimated probabilities.

2. The method of claim 1, further comprising, after step (c), automatically reranking the findings in the list of findings as a function of the likelihood that the finding can disambiguate among the plurality of medical conditions changing as a result of changes in the list of findings specified by the user as being present or absent in the patient.

3. The method of claim 1, wherein step (c) further comprises identifying in the physical computing device one or more findings not identified from the electronic patient information as being relevant to diagnosis of the patient.

4. The method of claim 1, further comprising displaying mentions of one of the flagged findings from the electronic patient information.

5. The method of claim 4, wherein step (b) comprises aggregating multiple mentions of one of the flagged findings.

6. The method of claim 4, further comprising eliminating duplicates of the same mention of one of the flagged findings prior to the displaying.

7. The method of claim 1, step (b) comprises:
(i) processing of numeric data to determine percentiles over time; or
(ii) processing clinical notes to identify contextual information for flagged findings.

8. The method of claim 1, wherein step (b) comprises:
(i) displaying the flagged findings in an integrated list with other findings not flagged as being in the electronic patient information; and/or
(ii) displaying only the flagged findings in a standalone list.

9. The method of claim 1, wherein step (c) comprises inputting in the physical computing device the onset timing and/or the timing of disappearance, of the finding.

10. The method of claim 1, wherein in step (b) the flagged findings are identified using natural language processing (NLP) of the electronic patient information, either in real time or in advance, and/or using keyword searching of the electronic patient information.

11. The method of claim 10, wherein ontology codes identified as being present in the electronic patient information are matched to one or more findings in the list of findings.

12. The method of claim 11, wherein one or more ontology codes identified as being present in the electronic patient information are not matched to any findings in the list of findings.

13. The method of claim 11, wherein at least one ontology code is matched to more than one finding.

14. The method of claim 11, wherein an ontology code from a parent, sibling, and/or child concept is matched to the one or more findings.

15. The method of claim 11, wherein ontology codes from more than one ontology are matched to the one or more findings.

16. The method of claim 10, wherein the flagged findings are identified using natural language processing (NLP) of the electronic patient information, either in real time or in advance, and using keyword searching of the electronic patient information.

17. The method of claim 10, wherein the keyword searching comprises use of synonyms and/or abbreviations.

18. The method of claim 1, further comprising prior to step (c) displaying contextual information from the electronic patient information about each flagged finding.

19. The method of claim 18, wherein the contextual information allows for the determination of presence, absence, onset timing and/or the timing of disappearance of the flagged finding.

20. The method of claim 18, wherein the contextual information comprises text from the electronic patient information.

21. The method of claim 1, further comprising outputting a findings list useful in diagnosis.

22. The method of claim 21, wherein the findings list comprises ontology codes in human readable and/or machine-readable formats.

23. The method of claim 1, further comprising outputting a Return of Results report or saving a report in the electronic patient information.

24. The method of claim 1, wherein the electronic patient information comprises dictation or an electronic health record.

25. The method of claim 1, further comprising generating the pertinence of the findings in the list of findings and displaying the findings with an indicator of pertinence or outputting the list of findings ranked by pertinence.

26. The method of claim 1, where ranking the not-specified findings comprises weighting the likelihood that a finding can disambiguate between a plurality of medical conditions by a factor representative of a possibility that a disease can be treated effectively.

27. The method of claim 1, wherein the findings comprise genetic sequencing information associated with the patient comprising identification of one or more genetic variants, and for each of the one or more genetic variants, a measure of zygosity for the patient, wherein for each of said one or more genetic variants, a severity score is provided in the plurality of genetic findings or the computing device generates said severity score, and wherein estimated probabilities of the candidate diseases are generated using the severity scores for each of the one or more genetic variants.

28. The method of claim 1, wherein step (b) comprises importing notes, chart values, lab results, and/or metadata about the context, date, and clinicians making the observation.

29. The method of claim 1, further comprising testing for a finding not identified in the electronic patient information.

30. The method of claim 1, further comprising treating the patient based on the estimated probabilities of the medical conditions.

31. The method of claim 1, wherein the one or more findings specified as being present or absent comprises at least one flagged finding.

32. A non-transitory computer readable medium having stored therein
(a) a plurality of candidate medical conditions;
(b) a list of findings, each of which is representative of clinical information about the medical conditions; and
(c) instructions for causing one or more processors to execute steps comprising:
(i) ranking findings in the list of findings as a function of the likelihood that the finding can disambiguate among the plurality of medical conditions;
(ii) identifying one or more findings from an output of a search of electronic patient information of a patient and flagging those findings in the list of findings;
(iii) displaying an indicator to a user for any flagged finding in the list of findings;
(iv) providing an interface for the user to specify one or more flagged or not-flagged findings as being present or absent in the patient;
(v) generating estimated probabilities of the medical conditions using the one or more findings specified as being present or absent; and
(vi) outputting a candidate disease list of the medical conditions ranked by highest estimated probabilities.

33. The medium of claim 32, wherein the instructions further comprise automatically reranking the findings in the list of findings as a function of the likelihood that the finding can disambiguate among the plurality of medical conditions changing as a result of changes in the list of findings specified by the user as being present or absent in the patient.

34. The medium of claim 32, wherein the instructions further comprise displaying to the user mentions of one of the flagged findings from the electronic patient information.

35. The medium of claim 34, wherein the instructions further comprise aggregating multiple mentions of one of the flagged findings.

36. The medium of claim 34, wherein the instructions further comprise eliminating duplicates of the same mention of one of the flagged findings prior to the displaying.

37. The medium of claim 32, wherein the search of the electronic patient information comprises processing of numeric data to determine percentiles over time or processing clinical notes to identify contextual information for findings.

38. The medium of claim 32, wherein (iii) comprises displaying the flagged findings in an integrated list with other findings not flagged as being in the electronic patient information; and/or displaying only the flagged findings in a standalone list.

39. The medium of claim 32, wherein the search uses natural language processing (NLP) of the electronic patient information, either in real time or in advance and/or using keyword searching of the electronic patient information.

40. The medium of claim 32, further comprising a set of curated ontology codes from the search of the electronic patient information that match with one or more findings in the list of findings.

41. The medium of claim 40, wherein the set of curated ontology codes comprises codes from more than one ontology.

42. The medium of claim 32, wherein the instructions further comprise searching the electronic patient information for keywords and/or abbreviations to identify findings.

43. The medium of claim 32, wherein the instructions further comprise displaying contextual information from the electronic patient information about each flagged finding.

44. The medium of claim 32, wherein the instructions further comprise outputting a findings list useful in diagnosis; outputting a Return of Results report; and/or saving a report in the electronic patient information.

45. The medium of claim 32, wherein the instructions further comprise generating the pertinence of the findings in the list of findings and displaying the findings with an indicator of pertinence or outputting the list of findings ranked by pertinence.

46. The medium of claim 32, wherein ranking the not-specified findings comprises weighting the likelihood that a finding can disambiguate between a plurality of medical conditions by a factor representative of a possibility that a disease can be treated effectively.

47. The medium of claim 32, wherein the findings comprise genetic sequencing information associated with the patient comprising identification of one or more genetic variants, and for each of the one or more genetic variants, a measure of zygosity for the patient, wherein for each of said one or more genetic variants, a severity score is provided in the plurality of genetic findings or the instructions further comprise generating the severity score, and wherein estimated probabilities of the candidate diseases are generated using the severity scores for each of the one or more genetic variants.

48. A system comprising a physical computing device comprising one or more processors, a network communication interface, and one or more computer readable memories having stored therein a plurality of candidate medical conditions; a list of findings, each of which is representative of clinical information about the medical conditions; and instructions that when executed by the one or more processors cause the system to
(i) rank findings in the list of findings as a function of the likelihood that the finding can disambiguate among the plurality of medical conditions;
(ii) identify findings in an output of a search of electronic patient information of a patient and flag those findings in the list of findings;
(iii) display an indicator for any flagged finding in the list of findings;
(iv) provide an interface for a user to specify one or more flagged or not-flagged findings as being present or absent in the patient;
(v) generate estimated probabilities of the medical conditions using the one or more findings specified as being present or absent; and
(vi) output a candidate disease list of the medical conditions ranked by highest estimated probabilities.

49. The system of claim 48, wherein the instructions further cause the system to automatically rerank the findings in the list of findings as a function of the likelihood that the finding can disambiguate among the plurality of medical conditions changing as a result of changes in the list of findings specified by the user as being present or absent in the patient.

50. The system of claim 48, wherein the instructions further cause the system to display to the user mentions of one of the flagged findings from the electronic patient information.

51. The system of claim 50, wherein the instructions further cause the system to aggregate multiple mentions of one of the flagged findings.

52. The system of claim 50, wherein the instructions further cause the system to eliminate duplicates of the same mention of one of the flagged findings prior to the displaying.

53. The system of claim 48, wherein the search of the electronic patient information comprises processing of numeric data to determine percentiles over time or processing clinical notes to identify contextual information for findings.

54. The system of claim 48, wherein (iii) comprises displaying the flagged findings in an integrated list with other findings not flagged as being in the electronic patient information; and/or displaying only the flagged findings in a standalone list.

55. The system of claim 48, wherein the search uses natural language processing (NLP) of the electronic patient information, either in real time or in advance and/or using keyword searching of the electronic patient information.

56. The system of claim 48, wherein the one or more computer readable memories has further stored therein a set of curated ontology codes from the search of the electronic patient information that match with one or more findings in the list of findings.

57. The system of claim 56, wherein the set of curated ontology codes comprises codes from more than one ontology.

58. The system of claim 48, wherein the instructions further cause the system to search the electronic patient information for keywords and/or abbreviations to identify findings.

59. The system of claim 48, wherein the instructions further cause the system to display contextual information from the electronic patient information about each flagged finding.

60. The system of claim 48, wherein the instructions further cause the system to output a findings list useful in diagnosis; output a Return of Results report; and/or save a report in the electronic patient information.

61. The system of claim 48, wherein the instructions further cause the system to generate the pertinence of the findings in the list of findings and display the findings with an indicator of pertinence or output the list of findings ranked by pertinence.

62. The system of claim 48, wherein ranking the not-specified findings comprises weighting the likelihood that a finding can disambiguate between a plurality of medical conditions by a factor representative of a possibility that a disease can be treated effectively.

63. The system of claim 48, wherein the findings comprise genetic sequencing information associated with the patient comprising identification of one or more genetic variants, and for each of the one or more genetic variants, a measure of zygosity for the patient, wherein for each of said one or more genetic variants, a severity score is provided in the plurality of genetic findings or the instructions further cause the system to generate the severity score, and wherein estimated probabilities of the candidate diseases are generated using the severity scores for each of the one or more genetic variants.

64. The system of claim 48, wherein the electronic patient information comprises dictation or an electronic health record.
